(19) United States
(12) Patent Application Publication
(10) Pub. No.: US 2020/0221841 A1
(43) Pub. Date: Jul. 16, 2020

(54) FOLDABLE PACKAGE CONTAINING DIVERSE ITEMS

(71) Applicant: Aasef Shafik, Arlington, VA (US)

(72) Inventor: Aasef Shafik, Arlington, VA (US)

(21) Appl. No.: 16/245,227

(22) Filed: Jan. 10, 2019

(51) Int. Cl. A45C 11/00; A61F 6/00; B65D 75/04; A45C 13/02; B31B 70/26

(52) U.S. Cl. CPC A45C 11/008; A61F 6/005; B65D 75/04; B31B 2160/10; B31B 70/26; A45C 2011/007; A45C 13/02

(57) ABSTRACT

A foldable package for containing multiple diverse items for convenient access to the items, said package having first and second pocket parts with front surfaces, back surfaces, top and bottom edges, and opposite end edges. The pocket parts are foldably joined to each other along a spine and are pivotable about the spine from an open position to a closed position with one pocket part overlying the other so that the front surfaces are in confronting juxtaposed relationship. At least one pocket is formed on the front surface of each pocket part, the pockets having open upper edges through which items may be inserted into and removed from the pockets. A closure flap on one of the pocket parts is foldable over an adjacent edge of the other pocket part to secure the pocket parts in closed position.

### TECHNICAL FIELD

This invention relates to packaging for convenient access to multiple diverse items, and more particularly to a foldable package that in one embodiment contains chewing gum and breath mints, and in another embodiment, contains chewing gum, breath mints and a condom. Either embodiment conveniently fits in a pocket or purse and can be made disposable or reusable.

### BACKGROUND ART

Persons frequently chew gum or use breath mints to freshen their breath, especially when engaging in social interaction with other persons. Many different flavors and forms of chewing gum and breath mints are sold to satisfy the tastes and desires of different persons.

Chewing gum typically is packaged in individual separate containers for different flavors, e.g., Mint, Spearmint, Peppermint, Wintergreen, etc., and for different forms, e.g., bars, pellets, slabs, etc. Similarly, breath mints are typically packaged in individual separate containers for different flavors and forms. Therefore, the consumer, when buying chewing gum or breath mints generally must buy a separate package for each flavor and each form.

U.S. Pat. No. 6,644,488 is exemplary of a combination package for chewing gum and breath mints that obviates the need for buying a separate package for each, but this package is relatively complicated and expensive in construction.

U.S. Pat. No. 7,563,468 discloses a chewing gum package for pieces of gum that are not individually wrapped, wherein at least one gum disposal sheet is provided in the package to wrap gum in for disposal after it has been chewed.

Published U.S. patent application 2013/0189393 discloses a folding package for containing multiple flavors of bars of chewing gum.

Social interaction can involve sexual intercourse. Prophylactics, more commonly known as condoms, are typically utilized to reduce the risk of pregnancy and the spread of sexually transmitted diseases. As such, sexually active individuals usually carry on their person conventional individually packaged condoms.

U.S. Pat. No. 4,741,434 discloses a keyholder having a case for holding a condom package. This keyholder condom case does not provide easy access as pins must first be removed to use the condom package, followed by opening of the package to access the condom container.

U.S. Pat. No. 5,316,136 discloses a case for housing packaged condoms. The case includes upper and lower lids and a pair of holes for receiving two separate keys that simultaneously disengage a pair of locking mechanisms within the case. This packaged condom housing arrangement is mechanically intricate and inconvenient.

Applicant is not aware of any prior package of convenient size and construction that contains multiple types of breath fresheners, i.e. chewing gum and breath mints. and that may be easily carried in a pocket or purse, nor is applicant aware of any prior package that contains at least one condom and at least one type of breath freshener for easy access and that may be easily carried in a pocket or purse.

It would be advantageous to have a convenient and easy to use package containing breath fresheners of multiple flavors and forms and that may be easily carried in a pocket or purse, and further to have a convenient and easy to use package that may be easily carried in a pocket or purse and that contains at least one type of breath freshener and at least one condom.

### DISCLOSURE OF INVENTION

The present invention is a convenient and easy to use package that may be easily carried in a pocket or purse and in a first embodiment contains breath fresheners of multiple flavors and forms, and in a second embodiment, contains at least one type of breath freshener and at least one condom.

More particularly, the invention is a foldable package for containing multiple diverse items for convenient access to the items, said package having first and second pocket parts with front surfaces, back surfaces, top and bottom edges, and opposite end edges. The pocket parts are foldably joined to each other along a spine and are pivotable about the spine from an open position to a closed position with one pocket part overlying the other so that the front surfaces are in confronting juxtaposed relationship. At least one pocket is formed on the front surface of each pocket part, the pockets having open upper edges through which items may be inserted into and removed from the pockets. A closure flap on one of the pocket parts is foldable over an adjacent edge of the other pocket part to secure the pocket parts in closed position.

In a first embodiment, the package comprises two pocket parts foldably joined together along a spine, with pockets on the confronting faces of the pocket parts. The pockets are defined by front panels overlying a back panel. In a preferred construction, a single pocket containing a condom is formed on the front of one of the pocket parts, with the upper edge of the single pocket angled to provide easy access to the condom held in the pocket. A pair of pockets are formed on the front of the opposite pocket part, with either chewing gum or breath mints contained in both pockets, or chewing gum in one pocket and breath mints in the other pocket. The upper edges of the pair of pockets are vertically offset from one another and extend generally parallel to the top and bottom edges of the package. A closure flap on the top edge of one of the pocket parts is folded over the upper edges of both pocket parts when the package is closed to hold the package closed and prevent loss of product from the pockets during storage of the package. A second closure tab may optionally be provided on the outer side edge of one of the pocket parts to help hold the package closed.

A blank for erecting the first embodiment of foldable package comprises first and second side-by-side rectangularly shaped back panels joined to each other along a first fold line at adjacent side edges thereof. A first rectangularly shaped front panel is joined along a second fold line to a side edge of the second back panel opposite the first fold line, and bands of adhesive are along the bottom and outer end edges of the first front panel. A second rectangularly shaped front panel is joined along a third fold line to a bottom edge of the second back panel, and bands of adhesive are along opposite side edges of the second front panel. A third front panel having an inclined upper edge is joined along a fourth fold line to the side edge of the first back panel opposite the first fold line, and said third front panel has bands of adhesive along the bottom and outer end edge thereof. A closure flap is foldably joined along a fifth fold line to a top edge of the second back panel, and a band of adhesive is along the edge of the closure flap opposite the fifth fold line.

In a second embodiment, the package also comprises two pocket parts foldably joined together along a spine, but in this embodiment a single first pocket is formed on the front of one of the pocket parts for receiving a first product, and a single second pocket is formed on the front of the other pocket part for receiving the same or a different product. In a preferred construction, the pockets are defined by a front panel overlying a back panel, with the upper edges of the pockets extending at an angle to the top and bottom edges of the package to provide easy access to product in the pockets. A closure 100 flap on the top edge of one of the pocket parts is folded over the upper edges of the pocket parts and releasably attached to the opposite pocket part when the package is closed. In the present invention, both pockets contain either chewing gum or breath mints, or one of the pockets contains chewing gum and the other pocket contains breath mints.

A blank for erecting the second embodiment of foldable package comprises first 105 and second side-by-side rectangularly shaped back panels joined to each other along a first fold line at adjacent first side edges. A first front panel is joined along a second fold line to a second side edge of the first back panel opposite the first fold line. The first front panel has an inclined upper edge and bands of adhesive along bottom and outer end edges. A second front panel having an inclined upper edge and bands of adhesive are 110 along bottom and outer end edges thereof is joined along a third fold line to a second side edge of the second back panel opposite the first side edge. A closure flap is foldably joined along a third fold line to a top edge of the first back panel, said closure flap having a band of adhesive along an edge thereof opposite said third fold line.

### BRIEF DESCRIPTION OF THE DRAWINGS

The foregoing, as well as other objects and advantages of the invention, will become apparent from the following detailed description when considered in conjunction with the accompanying drawings, wherein like reference characters designate like parts throughout the several views, and wherein:

FIG. 1 is a plan view of a first embodiment of the package of the invention, shown in a closed position as it would be stored in a pocket or purse.

FIG. 2 is a fragmentary, partially opened, isometric view of the package of FIG. 1.

FIG. 3 is a plan view of the package of FIG. 1, shown in fully opened position for access to the product contained therein.

FIG. 4 is a plan view of the blank for making the package of FIGS. 1-3.

FIG. 5 is a plan view of a second embodiment of the package, with the package shown in opened position.

FIG. 6 is an isometric view of the package of FIG. 5, with the package shown in closed position as it would be stored in a pocket or purse.

FIG. 7 is a plan view of the blank for making the package of FIGS. 5 and 6.

### BEST MODES FOR CARRYING OUT THE INVENTION

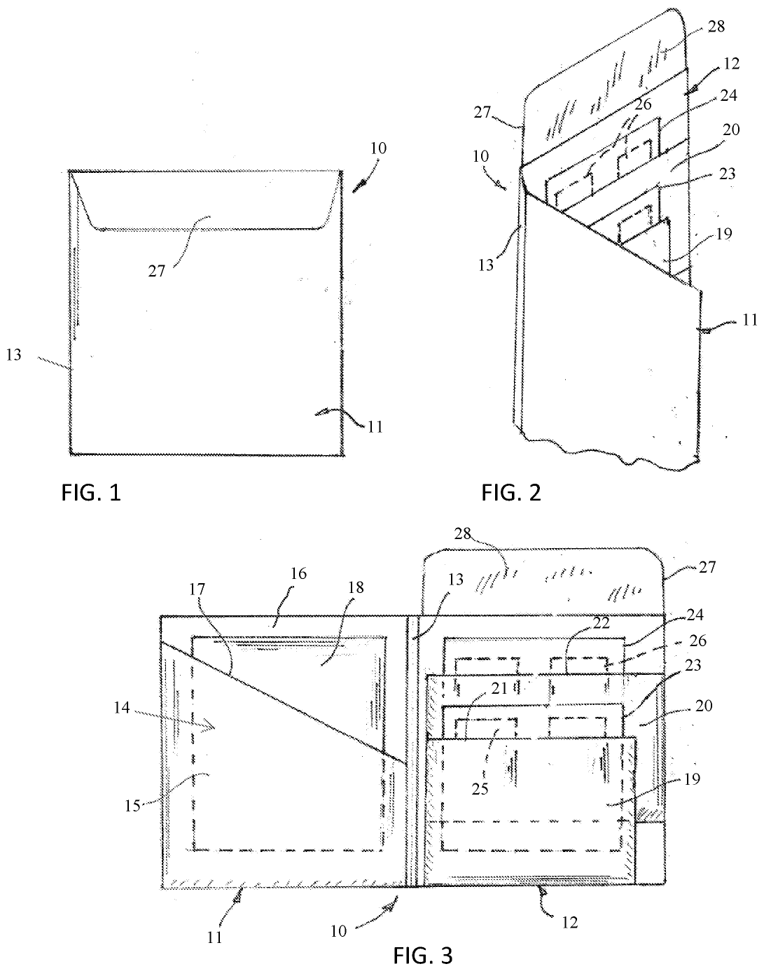

A first embodiment of package according to the invention is indicated generally at 10 in FIGS. 1-3. In this embodiment, the package comprises first and second pocket parts 11 and 12, respectively, foldably joined together along a spine 13 so that the pocket 135 parts have confronting faces.

A single pocket 14 defined by a front panel 15 overlying a back panel 16 is formed on the confronting face of pocket part 11 and has an angled open upper edge 17. A condom 18 is contained in pocket 14 so that an upper end portion of the condom is exposed above the angled open upper edge of the pocket to provides easy access to the 140 condom held in the pocket.

A pair of pockets 19 and 20 having open upper edges 21 and 22, respectively, are formed on the confronting or inner face of pocket part 12, with pocket 19 overlying a portion of pocket 20. The upper edges 21 and 22 of the pockets 19 and 20 are vertically offset from one another and extend generally parallel to the top and bottom edges of the 145 package.

Packages 23 and 24 of either chewing gum or breath mints 25 and/or 26, as desired by the individual user, are contained in both pockets, or chewing gum (e.g. 25) is contained in one pocket and breath mints (e.g. 26) is contained in the other pocket.

A closure flap 27 on the top edge of one of the pocket parts (pocket part 12 in 150 the particular example shown) has a releasable adhesive 28, or other suitable releasable fastening means, on one surface thereof so that when the pocket parts 11 and 12 are folded about spine 13 into closed position as shown in FIG. 1 and the flap 27 is folded over the upper edges of both pocket parts, the flap will hold the package closed and prevent loss of product from the pockets during storage and use of the package.

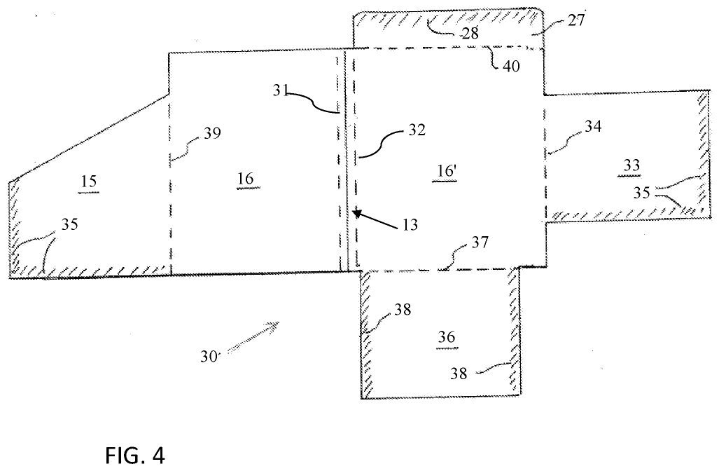

A blank 30 for making the package of FIGS. 1-3 is shown in FIG. 4. Back panel 16 that forms the back of pocket 14 is foldably joined along spaced parallel fold lines 31 and 32 in spine 13 to back panel 16′, which, as later explained, forms the back or at least part of the back of pockets 19 and 20 in pocket part 12.

The pockets 19 and 20 in pocket part 12 are formed by first folding front panel 33 forwardly about fold line 34 into overlying relationship to back panel 16′ and securing it to panel 16′ with bands 35 of adhesive along the free end edge and bottom edge of the panel 33. The upper edge of panel 33 remains unattached to panel 16′ to provide an opening for placing product into and removing it from the pocket 20 formed by front and back panels 33 and 16′.

Pocket 19 is then formed by folding panel 36 upwardly about fold line 37 into overlying relationship with portions of back panel 16′ and previously folded panel 33 and securing it in place with bands of adhesive 38 along opposite side edges of panel 36. The top edge of panel 36 remains unattached to provide an opening for placing product into and removing it from pocket 19.

Single pocket 14 is formed by folding panel 15 inwardly about fold line 39 into overlying relationship to panel 16 and securing it in place with bands of adhesive 35 along the outer end and bottom edges of panel 15.

Closure flap 27 is foldably joined along fold line 40 to the upper edge of back panel 16′.

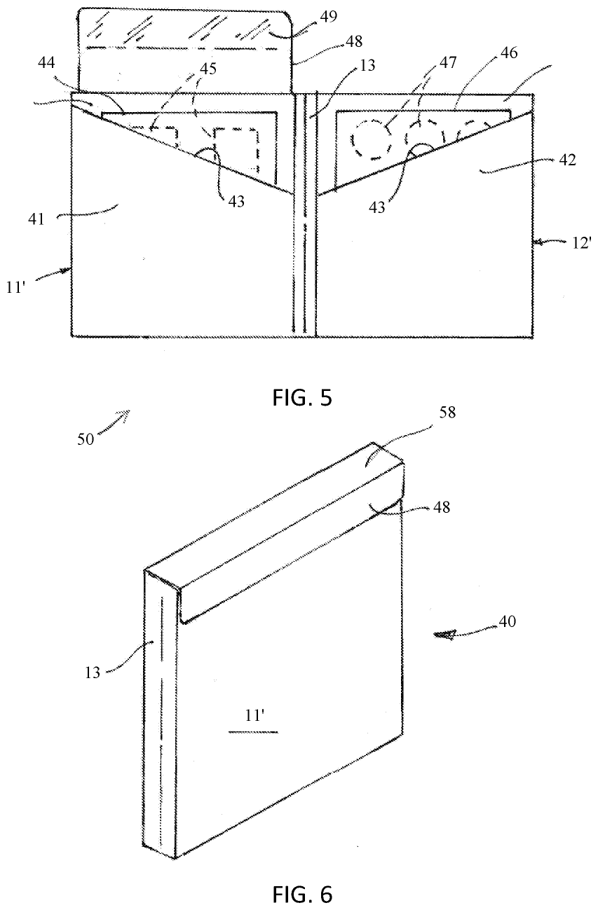

A package 50 according to a second embodiment is shown in FIGS. 5 and 6. This package also comprises two pocket parts 11′ and 12′ foldably joined together along a spine 13, with a single first pocket 41 in pocket part 11′ and a single second pocket 42 in pocket part 12′. The pockets have angled open upper edges 43 so that an upper portion of products contained in the pockets projects above the upper edge of the respective 180 pockets and is easily accessible. The products may comprise a package 44 of chewing gum 45 in one pocket and a package 46 of breath mints 47 in the other pocket, or the same product, i.e. either chewing gum or breath mints, may be contained in both pockets.

A closure flap 48 is foldably joined to the upper edge of one of the pocket parts 11′ or 12′ (11′ in the example shown), and a releasable adhesive 49 or other suitable 185 fastening means is on the flap for holding it in closed position when the pocket parts are folded into overlying relationship with one another and the flap is folded over the upper edges and secured as shown in FIG. 6.

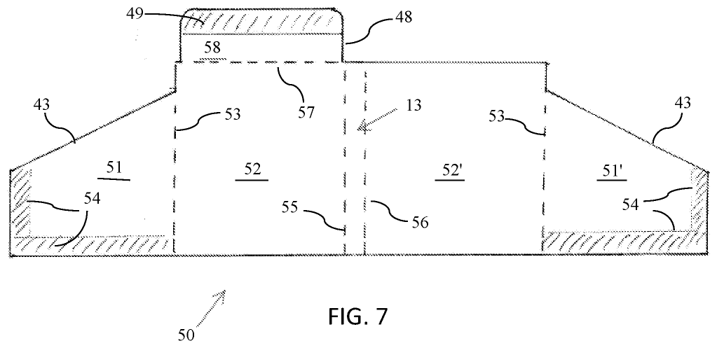

A blank 50 for making the package of FIGS. 5 and 6 is shown in FIG. 7. The pockets 41 and 42 are defined by front panels 51 and 51′, respectively, foldably joined to 190 back panels 52 and 52′ along fold lines 53 so that panels 51 and 51′ can be folded into overlying relationship with respective back panels 52 and 52′, with the upper edges 43 of the pockets extending at an angle to the top and bottom edges of the package to provide easy access to product in the pockets. Bands 54 of adhesive are on the outer end and bottom edges of panels 51 and 51′ to hold the panels 51 and 51′ in closed position when 195 the blank is assembled. Back panels 52 and 52′ are joined to the spine 13 along spaced apart fold lines 55 and 56.

The closure flap 48 is joined along fold line 57 to the upper edge of pocket part 52, and as seen best in FIGS. 6 and 7, a panel 58 extending between the fold line 57 and band of adhesive 49 has a width dimensioned to span the thickness of the package when it 200 is folded closed as shown in FIG. 6.

While particular embodiments of the invention have been illustrated and described in detail herein, it should be understood that various changes and modifications may be made in the invention without departing from the spirit and intent of the invention as defined by the appended claims.

What is claimed is:

1. A foldable package for containing multiple diverse items for convenient access to the items, said package comprising:
first and second pocket parts having front surfaces, back surfaces, top and bottom edges, and opposite end edges;
said pocket parts being foldably joined to each other along a spine so that said pocket parts are pivotable from an open position with the pocket parts lying on opposite sides of the spine in substantially parallel relationship to one another to a closed position with one pocket part overlying the other so that the front surfaces thereof are in confronting juxtaposed relationship;
at least one pocket formed on the front surface of each pocket part, said pockets having open upper edges through which items may be inserted into and removed from the pockets; and
a closure flap on one of said pocket parts, said closure flap being foldable over an adjacent edge of the other of said pocket parts to secure the pocket parts in said closed position.

2. The foldable package as claimed in claim 1, wherein:
there are two pockets on the front surface of one of said pocket parts, and a single pocket on the front surface of the other said pocket part.

3. The foldable package as claimed in claim 2, wherein:
the open upper edges of said two pockets are parallel to the top edge of said one pocket part and are spaced different distances from said top edge.

4. The foldable package as claimed in claim 3, wherein:
the open upper edge of said single pocket extends at an angle relative to the top edge of the pocket part.

5. The foldable package as claimed in claim 4, wherein:
a breath freshener is contained in said two pockets; and
a condom is contained in said single pocket.

6. The foldable package as claimed in claim 1, wherein:
there is a single pocket on the front surface of each of said pocket parts.

7. The foldable package as claimed in claim 6, wherein:
the open upper edge of each said single pocket extends at an angle relative to the top edge of the respective pocket part.

8. The foldable package as claimed in claim 7, wherein:
the closure flap is on the top edge of one of said pocket parts, said closure flap having a width to cover the top edge of the other of said pocket parts when the pocket parts folded into their closed position.

9. The foldable package as claimed in claim 8, wherein:
a breath freshener is contained in each of said single pockets.

10. A blank for erecting a foldable package for containing multiple diverse items for convenient access to the items, said blank comprising:
first and second side-by-side rectangularly shaped back panels joined to each other along a first fold line at adjacent side edges thereof;
a first front panel joined along a second fold line to a side edge of said second back panel opposite said first fold line, said first front panel being rectangularly shaped;
bands of adhesive along the bottom and outer end edges of said first front panel;
a second front panel joined along a third fold line to a bottom edge of said second back panel, said second front panel being rectangularly shaped;
bands of adhesive along opposite side edges of said second front panel;
a third front panel joined along a fourth fold line to the side edge of said first back panel opposite said first fold line, said third front panel having an inclined upper edge;
bands of adhesive along the bottom and outer end edge of said third front panel;
a closure flap foldably joined along a fifth fold line to a top edge of said second back panel; and
a band of adhesive along the edge of said closure flap opposite said fifth fold line.

11. A blank for erecting a foldable package for containing multiple diverse items for convenient access to the items, said blank comprising:
first and second side-by-side rectangularly shaped back panels joined to each other along a first fold line at adjacent first side edges thereof;
a first front panel joined along a second fold line to a second side edge of said first back panel opposite said first fold line, said first front panel having an inclined upper edge and bands of adhesive along bottom and outer end edges thereof;
a second front panel joined along a third fold line to a second side edge of said second back panel opposite said first side edge, said second front panel having an inclined upper edge and bands of adhesive along bottom and outer end edges thereof; and
a closure flap foldably joined along a third fold line to a top edge of said first back panel, said closure flap having a band of adhesive along an edge thereof opposite said third fold line.
